(19) United States
(12) Patent Application Publication
(10) Pub. No.: US 2026/0114834 A1
(43) Pub. Date: Apr. 30, 2026

(54) IMAGE QUALITY BY USING AN INDIRECTLY IRRADIATED DETECTOR SURFACE IN X-RAY IMAGING

(71) Applicant: Siemens Healthineers AG, Forchheim (DE)

(72) Inventors: Peter Bartl, Neunkirchen am Brand (DE); Niko Köster, Neunkirchen am Brand (DE); Robert Brauweiler, Baiersdorf (DE); Jens Bialkowski, Neunkirchen am Brand (DE); Stefan Söllradl, Nürnberg (DE)

(21) Appl. No.: 19/359,340

(22) Filed: Oct. 15, 2025

(30) Foreign Application Priority Data
Oct. 24, 2024 (DE) 10 2024 210 255.5

(51) Int. Cl. A61B 6/00; A61B 6/03

(52) U.S. Cl. CPC A61B 6/542; A61B 6/032

(57) ABSTRACT

A method for carrying out an X-ray imaging process based on X-ray radiation includes: detecting a primary radiation overlaid with secondary radiation in a region of a detector directly irradiated by the X-ray radiation; detecting a secondary radiation in an outer region of the detector outside of the directly irradiated region; automatically determining an irradiation parameter dependent upon the detected primary radiation and the detected secondary radiation; and carrying out the X-ray imaging based on the irradiation parameter.

The present patent document claims the benefit of German Patent Application No. 10 2024 210 255.5, filed Oct. 24, 2024, which is hereby incorporated by reference in its entirety.

### TECHNICAL FIELD

The present disclosure relates to a method for carrying out an X-ray imaging process based on X-ray radiation by detecting a primary radiation overlaid with secondary radiation in a region of a detector directly irradiated by the X-ray radiation. The present disclosure further relates to a computer program, an image processing apparatus, and an X-ray based imaging modality.

### BACKGROUND

In X-ray based imaging, in particular in medical imaging, scattered radiation may be a significant cause of a deterioration of image quality. Therefore, in the use of imaging methods such as computed tomography and/or fluoroscopy, the correction of scattered radiation may play a decisive role in image processing and/or the optimization of the workflow.

In trans-irradiation techniques such as fluoroscopy or a (serial) radiography, an X-ray source and a detector are used, wherein the object is situated between the X-ray source and the detector. Due to the partial absorption of the X-ray radiation in the object, which may vary depending upon the material, the directly transmitted X-ray radiation (designated “primary radiation” herein) may be representative of the internal structure of the object. Due to scattering of the radiation, however, scattered radiation (also designated “secondary radiation” herein) also reaches the detector, as a result of which the image quality is impaired. A collimator may be used in order to irradiate only a particular region of an object, e.g., a portion of a patient. In many cases, due to the collimator, only a subregion of the detector is irradiated. The collimator may reduce the radiation burden on the object and also the quantity of scattered radiation. However, some scattered radiation, in particular, radiation scattered inside the object, may still reach the aforementioned subregion of the detector and therefore impairs the image quality.

In computed tomography (CT), a primary X-ray beam generated by an X-ray source is directed onto an object in an examination region and is detected by an X-ray detector on the opposite side of the examination region. Depending upon the makeup of the object, parts of the primary X-ray beam are absorbed, which leads to an object-dependent attenuation. Based on the attenuation of the primary X-ray beam, structures of the object may be determined. By way of a variation of the projection angle of the primary X-ray beam onto the object, a three-dimensional image of the object may be generated. However, part of the X-ray radiation is scattered, which may result in the scattered radiation being detected by the X-ray detector. Scattered radiation detected by the CT detector may itself lead to image artifacts and therefore to a diminished image quality.

A measure for solving the problem of scattered radiation in CT imaging, fluoroscopy or a (serial) radiography may be an anti-scatter grid. Anti-scatter grids may be based upon the assumption that the angle of incidence of the primary X-ray beam on the detector is constant for every projection angle. While anti-scatter grids may be a useful measure in certain third generation CT systems with a rotating gantry, in the case of a CT scanner geometry with distributed, in particular, static non-rotating X-ray sources, they may not be useful. With distributed X-ray sources, the orientation of the source and the detector and thus the angle of incidence of the X-rays within the primary X-ray beam is different for each source of the distributed source field. The assumption that the angle of incidence of the primary X-ray beam onto the detector is constant for every projection angle is therefore no longer correct, which may have the result that corresponding anti-scatter grids cannot be implemented. Accordingly, scattered radiation may represent a problem, in particular, in the case of scatter geometries with distributed X-ray sources.

In order to tackle the problem of scattered radiation in CT, it has been proposed to provide a scatter estimation and correction by way of simulation of the physical process of X-ray interaction, for example, by Monte Carlo simulations and/or using scatter kernels, in order to estimate the scattered radiation directly for the primary X-ray beam. Corresponding correction and estimation methods are described, for example, by Rührnschopf, E. P., & Klingenbeck, K. in “A general framework and review of scatter correction methods in x-ray cone-beam computerized tomography” (2011), Part 1: Scatter Compensation Approaches. Medizinische Physik, 38(7), 4296-4311; and in Part 2:“Ansätze zur Streuschätzung” [Approaches to Scatter Estimation]. Medizinische Physik, 38(9), 5186-5199. Although direct Monte Carlo-based methods have the potential to be highly accurate, these methods have the disadvantage that they require much computation power and time. This may prevent their direct use in clinical CT image reconstruction.

Image quality in X-ray imaging is therefore degraded by secondary/scattered radiation. Due to physical processes scattering the primary X-ray radiation, every trans-irradiated object (e.g., patient) generates secondary radiation that is registered on the X-ray detector in addition to the actual primary signal. In order to reduce the detection of secondary radiation, anti-scatter grids may be used. In two-dimensional imaging, these are primarily grids with lamellae that reduce the scatter signal orthogonally to the direction of their lamellae. Grids with a two-dimensional structure are also used for 3D imaging. Furthermore, in some clinical applications, by increasing the spacing from the scattering object (e.g., patient), the scattered radiation falling upon the detector is reduced (e.g., air-gap method).

### SUMMARY AND DESCRIPTION

The object of the present disclosure lies in solving at least some of the aforementioned problems, in particular, offering a possibility for reducing the influence of scattered radiation on image data from X-ray-based imaging.

This object is achieved by way of a method, a computer program, an image processing apparatus, and an X-ray-based imaging modality as described herein. The scope of the present disclosure is defined solely by the appended claims and is not affected to any degree by the statements within this summary. The present embodiments may obviate one or more of the drawbacks or limitations in the related art.

In one aspect, a method is provided for carrying out an X-ray imaging process based on X-ray radiation. The method includes: detecting a primary radiation overlaid with secondary radiation in a region of a detector directly irradiated by the X-ray radiation; detecting a secondary radiation in an outer region of the detector outside of the directly irradiated region; automatically determining an irradiation parameter dependent upon the detected primary radiation and the detected secondary radiation; and carrying out the X-ray imaging based on the irradiation parameter.

The detection of the primary radiation or the secondary radiation in a region of the detector means that the respective radiation is incident upon a relevant region of the detector and is detected there by one or more detector elements. The detection may also include a preprocessing of the detection signals. The automatic determination of an irradiation parameter dependent upon the detected primary radiation and the detected secondary radiation may include an automatic calculation and/or an automatic assignment. For example, the automatic determination may be implemented by an algorithm and, in particular, by an algorithm for machine learning.

Carrying out the X-ray imaging based on the irradiation parameter means, for example, that the irradiation parameter is set accordingly in an X-ray imaging modality. For example, the X-ray dose and/or a corresponding tube voltage and/or a tube current is set as the irradiation parameter in the imaging modality. Alternatively, however, the irradiation parameter may also signal the presence of a filter or an anti-scatter grid, etc.

An X-ray imaging modality for the X-ray imaging may have an examination region between an X-ray source and an X-ray detector. The examination region may be configured so that an object to be examined and/or imaged may be placed in the examination region. The object may be part of a larger object or a subject. A subject may be a person, such as a patient, or an animal. The object may be an anatomical part, (e.g., a limb or an organ), of the subject. The object may be any other type of object, for example, a baggage item. For example, a table, (e.g., a patient table), may be provided that may be situated at least partially within the examination region. The table may be suitable for placing the object and/or the person onto the table. The table may be mobile, for example, in order to move the person or the object that is part of the person into the examination region.

The X-ray detector (detector, for short) may include detector elements. Detector elements may be designated detector pixels or may be representative of detector pixels. The detector resolution and/or the detector size may be (e.g., partially) determined by the detector elements and their relative spacing from one another may be determined.

The (X-ray) imaging modality may include at least one X-ray source. Therefore, an imaging modality based upon X-ray radiation is an imaging modality that uses X-ray radiation for generating images. Examples of such imaging modalities may be computed tomography systems, fluoroscopes, and projection radiographs. The at least one X-ray source, in particular, the X-ray sources, may be configured so that the X-ray source emits the primary X-ray beam. An X-ray source may also be designated an X-ray generator. The at least one X-ray source may be directed toward the examination region and the X-ray detector, wherein at least a part of the X-ray detector is situated outside the examination region. For example, the imaging modality may be based upon an X-ray tube according to the prior art.

The directly irradiated region (hereinafter also designated subregion, for short) includes a part of the detection region of the X-ray detector. Therefore, a part of the X-ray detector (e.g., outer region) is not directly irradiated by the primary X-ray beam. “Direct irradiation” may be understood to mean that X-ray radiation that irradiates directly is transmitted directly through the object or past the object (e.g., dependent upon the size and shape of the object) to the detector. Scattered radiation, however, is not part of the primary X-ray beam that irradiates the subregion directly. Some scattered radiation may also reach the directly irradiated subregion. Therefore, the detected signal from the part of the detection region of the detector may be based upon the radiation of the primary X-ray beam (i.e., primary radiation) and the scattered radiation (i.e., secondary radiation). The primary X-ray beam may be an X-ray conical beam. The scattered radiation contains no positional information about the object being investigated in the spatial frequency domain, which the detector pixels provide by way of their sampling. Therefore, a certain grouping together (e.g., binning) of the pixel signals of the scatter signal is useful under certain circumstances.

In the context of this disclosure, the X-ray radiation data that is recorded from the detector within the subregion is designated inner radiation data, i.e., radiation data that is detected within the subregion. The scattered radiation that is detected by the detector within the subregion may be designated data on the inner scattered radiation. In the context of this application, “X-ray radiation” in general may also be designated simply “radiation.”

Radiation may be scattered within the object or, in some cases, by other scatter fields. The data on the outer scattered radiation may be based upon the measurement of the entire detector region outside the directly irradiated subregion. Alternatively, the data on the outer scattered radiation may be based only on a part of the detector region outside the subregion. For example, the data on the outer scattered radiation may be based solely on the part of the detector region outside the subregion that is closest to the subregion.

Thus, with the same irradiation geometry and the same detector, the outer region in which the scattered radiation is detected may be selected to be different. Since the detector region outside the subregion is not directly irradiated by the primary X-ray beam, this outer detector region may only be reached by scattered radiation. Accordingly, it may be assumed that all the radiation data that reaches this outer detector region is substantially (e.g., more than 50%), in particular exclusively (e.g., 100%), scattered radiation. In the context of this disclosure, the X-ray radiation detected outside the subregion is designated “data on the outer scattered radiation.” The data on the inner radiation and the data on the outer scattered radiation may be detected substantially simultaneously and/or during the same measurement. It may also be possible to detect this data separately, for example, in sequential measurements. However, it may be more advantageous to detect the data simultaneously in order to save time and to reduce the X-ray dose applied.

According to one embodiment, the imaging modality is a computed tomography (CT) system, in particular, a static computed tomography system, wherein the X-ray detector includes an arrangement of detector elements that may be arranged at least partially around an examination region, wherein the directly irradiated subregion includes a subset of the detector elements, wherein the data on the outer scattered radiation is detected with at least some of the detector elements outside the directly irradiated subregion. The examination region of the computed tomography system may be configured so that the object may be placed in the examination region. The computed tomography system may include a gantry, wherein the gantry includes the at least one X-ray source and the detector elements of the X-ray detector. The examination region may be arranged, in particular, at least partially within the gantry. In certain examples, a static computed tomography system may be a computed tomography system with X-ray sources and detector elements that do not rotate during a computed tomography examination. The gantry may be configured so that it does not rotate about the examination region during a computed tomography scan. Accordingly, the X-ray detector may be a static X-ray detector. Advantageously, with a static computed tomography system, rotational forces may be avoided. The detector elements of the X-ray detector are at least partially arranged around the examination region. The arrangement of the detector elements partially around the examination region may be understood to mean that the detector elements are distributed only at parts of the circumference, for example, an annular circumference of the examination region and/or that there is a gap in the circumference of the examination region, wherein the gap has no or fewer detector elements than other parts of the circumference. The detector elements may be arranged, for example, curved and/or in a partially annular form. The detector elements may also be distributed over a circular or partially circular arrangement around the examination region. In certain examples, an X-ray detector may be used where the arrangement of the detector elements have an extent that may be at least 5% larger or at least 10% larger than the directly irradiated subregion of the detector. In particular, the detector elements may be distributed over an arrangement with an angle that is greater than the angle of the conical form of the primary X-ray beam. The detector elements may be distributed over 360°. In other words, the detector elements may be arranged entirely around the examination region.

In certain examples, the direction perpendicular to the arrangement of the detector elements of the CT system may be designated the axial direction. In particular, the axial direction may be perpendicular to a region that is generated by the arrangement of the detector elements. The axial direction may be the axial direction of a (e.g., partially) annular arrangement of the detector elements. The axial direction may correspond to a z-direction in Cartesian coordinates. A distribution in an axial direction may also exist if a general shape such as an annular or ring-shaped arrangement of detector elements is under consideration. A direction that extends perpendicularly to the axial direction may be designated a radial direction. In particular, the radial direction may be the direction from the center of the examination region to one of the detector elements. The radial direction may correspond to an x-, y-direction. Furthermore, a circumferential direction may be defined. The circumferential direction is a direction that follows the arrangement of the detector elements around the examination region. In particular, in an annular detector, the circumferential direction may follow the annular form of the ring.

The arrangement of the detector elements of the computed tomography system may include rows and/or columns of detector elements. Rows of detector elements may be detector elements arranged one behind the other in the circumferential direction.

Columns of detector elements may be detector elements arranged one behind the other in the axial direction. Therefore, the distribution of the detector elements may extend in the axial direction.

The computed tomography system may include at least one X-ray source, e.g., a plurality of X-ray sources. The at least one X-ray source, in particular, the X-ray sources, may be configured so that the X-ray source emits the primary X-ray beam. For example, distributed X-ray sources may be used in which a plurality of X-ray sources are distributed over a circular or partially circular arrangement around the examination region. The X-ray sources of the computed tomography system may be distributed around the examination region. The X-ray sources may be distributed over 360°. The X-ray sources may be part of an X-ray source field. The X-ray source field may be annular. The X-ray source field may be a stationary X-ray source field, in particular, according to the static computed tomography system. Therefore, the X-ray sources may be stationary during operation. In other words, in the case of a stationary computed tomography system, during a scan, the X-ray sources may not be rotated about the examination region. Both the X-ray sources of the computed tomography system and also the detector elements may be distributed around the examination region. The X-ray sources may be oriented toward the detector elements, in particular, the detector elements on the opposite side of the examination region. In particular, the X-ray sources, the examination region and the X-ray detector may be arranged such that the primary X-ray beam may reach the detector elements of the X-ray detector in a straight line through the examination region. A rotary sampling of the examination region may be achieved by selective activation of X-ray sources. For example, the X-ray sources may be selectively activated by way of electronic switching.

The subregion includes a subset of the detector elements. Therefore, some of the X-ray detector elements are not directly irradiated by the primary X-ray beam. “Direct irradiation” may be understood to mean that X-ray radiation that irradiates directly is transmitted directly through the object or past the object (for example, dependent upon the size and shape of the object) to the detector elements. Scattered radiation, however, is not part of the primary X-ray beam that irradiates the subregion directly. Scattered radiation may also reach the directly irradiated subregion. Therefore, the detected signal from the subset of detector elements may include radiation from the primary X-ray beam and scattered radiation. The primary X-ray beam may be a conical X-ray beam.

According to one embodiment, the subregion of the X-ray detector is a coherent arc-shaped portion of the arrangement of detector elements, in particular, corresponding to a conical angle of a conical primary X-ray beam. Therefore, the angle of the cone may define the subregion of the X-ray detector that is directly irradiated. The data on the outer scattered radiation may be based upon the measurement of all the detector elements outside the subregion. Alternatively, the data on the outer scattered radiation may be based on just a part of the detector elements outside the subregion. For example, the data on the outer scattered radiation may be based solely on the detector elements outside the subregion that are closest to the subregion.

According to one embodiment, the detector elements of the CT system are arranged in an annular form around the examination region, in particular, in the form of a 360° detector. A 360° detector is, in particular, an X-ray detector in which the detector elements are arranged round the examination region. A 360° detector may be advantageous since more radiation, (that is, from a larger overall detector surface), may be used for determining the scattered radiation within the subregion. With more scattered radiation information, the scatter correction may be determined more accurately. In particular, a 360° detector may supply information regarding the shape of the object and the scattering properties of the object. The scattering properties may contain information regarding the inner structure and the materials within the object that influence the scattering of the radiation.

According to one embodiment, the imaging modality is a fluoroscope that includes a collimator, wherein the collimation via the collimator defines the subregion on the X-ray detector. The fluoroscope may be a C-arm or may include a mobile C-arm. The fluoroscope may include an X-ray source and an X-ray detector. The X-ray source and the X-ray detector may be situated at opposite ends of the C-arm. The collimator may be situated between the X-ray source and the X-ray detector, in particular, in a straight line between the X-ray source and the X-ray detector. The direct line between the X-ray source and the X-ray detector may be defined in the context of this disclosure as the longitudinal direction. The collimator may be situated in front of the X-ray source. In particular, the collimator may be arranged between the X-ray source and the examination region. The collimator may be configured such that a collimation window may be adapted via the collimator. Optionally, the collimator may be set for this method such that the subregion on the detector that is directly irradiated by the X-ray radiation is smaller than the remaining region of the detector that is not directly irradiated by the X-ray radiation. The degree of collimation may depend upon the zoom format that is used for a particular transillumination examination. The collimation may be set automatically or manually. Although the collimation may prevent the X-ray radiation reaching outside the subregion, (i.e., outside the collimation window), directly onto the detector, scattered radiation or radiation scattered by the examination object (e.g., part of a patient) may still reach the region outside the subregion. Advantageously, by way of the use of the information from outside the subregion, a suitable estimate of the scattered radiation within the subregion may be achieved. This may lead to an improvement of the image processing and/or an optimization of the workflow.

In the case of a flat detector, the directly irradiated (sub)region may be a rectangular region of the detector surface and the outer region may be a frame or subframe around the directly irradiated subregion. The frame may be formed on all four sides of the rectangular region and the subframe, or on just three sides, two sides, or one side of the rectangular region. Alternatively, a circular collimator may be used such that a circular directly irradiated subregion and an outer region result that at least partially surrounds the subregion. Further alternatively, a rectangular collimator may also be inserted inclined, by which means, a directly irradiated subregion in the form of a parallelogram and an outer region result that at least partially surrounds the subregion. Furthermore, other collimator forms and/or orientations are possible with which corresponding directly irradiated subregions and outer regions result.

According to one embodiment, the estimation of the part of the inner radiation data attributable to the scattered radiation includes the application of a calculation model and/or an algorithm that derives the scattered radiation within the subregion from the data on the outer scattered radiation. Advantageously, the calculation model or the algorithm may be relatively simple, (i.e., may require relatively little calculation time), since based on the data provided on the outer scattered radiation, it is not necessary to simulate the scattering completely anew, but rather the data on the outer scattered radiation may be used as a basis.

According to one embodiment, the scattered radiation is determined within the subregion by interpolation from the data on the outer scattered radiation. For example, a linear interpolation from at least some of the data on the outer scattered radiation may be applied. A second-order or higher-order polynomial may be possible and/or necessary for the interpolation. The interpolation, (e.g., the linear interpolation), may be based upon the detector part, (e.g., the detector elements), outside the subregion that is closest to the subregion. In other words, the interpolation may be carried out based on the measured scattered radiation intensities of the detector part that lies just outside the irradiating primary X-ray beam. Advantageously, the application of an interpolation may offer a simple possibility for using the data on the outer scattered radiation for the determination of the scatter correction.

The X-ray imaging is carried out based on an irradiation parameter. In an embodiment, the irradiation parameter may relate to an X-ray dose. In this case, the X-ray dose is automatically determined dependent upon the detected primary radiation and possibly upon the detected secondary radiation (for example, by calculation, attribution, etc.).

According to a further embodiment, the X-ray dose for the X-ray imaging is regulated by way of an automatic dose regulation. A characteristic of the regulation is feedback. For example, the X-ray dose is regulated based on an image quality measure (e.g., contrast). For this purpose, a measured actual quality measure is compared with a corresponding target quality measure and the X-ray dose is changed until the target-actual deviation is a minimum according to selected quality criteria.

In a further embodiment, the irradiation parameter relates to a presence (and absence) of an anti-scatter grid or a filter in a beam path of the X-ray radiation and, for the automatic determination of the irradiation parameter, an effect of the anti-scatter grid and/or filter on the X-ray radiation is taken into account. In this case, the irradiation parameter is a binary variable that defines whether or not the anti-scatter grid or the filter is inserted into the beam path. For example, “1” means the presence of the anti-scatter grid and “0” means the absence of the anti-scatter grid (hereinafter also representing the filter) in the beam path of the X-ray radiation. Given the presence of the anti-scatter grid in the beam path, it may affect the X-ray radiation. In particular, the anti-scatter grid has an effect upon the ratio and/or the relationship between the primary radiation and the secondary radiation. This effect may be determined by measuring technology and therefrom, an information item may be obtained regarding whether the presence of the anti-scatter grid in the beam path is useful or not. For example, this information is obtained in that, based on the ratio between the primary radiation and the secondary radiation, an image quality is estimated and, if a corresponding image quality measure does not exceed or fall below a predetermined threshold value, the presence and/or absence of the anti-scatter grid is regarded as useful. Accordingly, as the irradiation parameter, the presence or absence of the anti-scatter grid is determined automatically, that is, recommended for further X-ray imaging. Thus, for example, an anti-scatter grid is disadvantageous for small objects since small objects generate little scattered radiation. In the case of larger objects, however, an anti-scatter grid may be advantageous. Furthermore, however, the use of the anti-scatter grid may also depend upon the material and it may therefore be determined automatically, dependent upon the material of the trans-irradiated object, whether the presence of the anti-scatter grid is useful. A corresponding irradiation parameter regarding the presence and/or absence may then be output. The presence of a filter may affect the spectrum and therefore the scattering and thus finally also the ratio between the primary radiation and the secondary radiation.

According to a further embodiment, the irradiation parameter is iteratively determined such that an image quality (for example, contrast) is optimized in the X-ray imaging. The acts of the method may thus be repeated multiple times in an iterative loop. The repetition may be ended if an image quality measure regarding the image quality has reached or exceeded a particular value. Thus, for example, the X-ray dose may be increased as an irradiation parameter until a particular image quality has been achieved. Alternatively, the X-ray dose may also be reduced until a particular image quality measure is reached and/or undershot. In the latter case, therefore, an optimization is carried out in relation to the radiation burden.

In a further embodiment, the irradiation parameter is determined automatically in that a ratio between the detected primary radiation and the detected secondary radiation in relation to an optimization criterion is optimized or maximized. As already indicated, the aim of the automatic determination of the irradiation parameter may be found in optimizing and/or maximizing (or minimizing) the ratio between the detected primary radiation and the detected secondary radiation. For example, the ratio may be controlled within a local or global maximum and/or minimum. Here, as in other examples (provided not stated otherwise), the “detected primary radiation” may be the primary radiation overlaid with the secondary radiation or the pure primary radiation determined, for example, by subtraction of an estimated secondary radiation component.

According to one further embodiment, for the automatic determination of the irradiation parameter, a pure component of the primary radiation may be determined in that the secondary radiation is interpolated for the directly irradiated region from the detected secondary radiation outside the directly irradiated region and the interpolated secondary radiation is subtracted from the detected overlaid primary radiation. In the present example, for the determination of the irradiation parameter, the pure component of the primary radiation is thus used and not the primary radiation overlaid with the secondary radiation. The interpolation of the secondary radiation may take place based on a single measurement value and/or pixel in the outer region of the detector where only the secondary radiation is detected. The interpolation may take place based on a plurality of sampling sites arranged around the directly irradiated region. For example, two sampling sites may be arranged on two opposite sides of the directly irradiated region. Thus, for instance, two mutually opposed frame portions that are part of the outer region of the detector and between which the directly irradiated region is arranged may be made use of for the interpolation. Naturally, the interpolation may also take place two-dimensionally based on correspondingly two-dimensional sampling sites. The secondary radiation and/or scattered radiation distribution interpolated in this way may be subtracted from the primary radiation (and/or primary radiation distribution) overlaid with secondary radiation in order to obtain the pure component of the primary radiation again.

In a further embodiment, the pure component of the primary radiation represents the control variable in automatic dose regulation. In this example, therefore, the X-ray dose is regulated and in this regulation, a certain setpoint value may be specified for the primary radiation. According to a further embodiment, when the X-ray imaging is carried out, an X-ray image is obtained only based on the pure component of the primary radiation. This means that the X-ray image without the effect of the secondary radiation is obtained. The X-ray image is therefore corrected with respect to the interfering component of the secondary radiation or scattered radiation. By way of such a correction, for example, the entire brightness range or black-and-white range is better utilized and thereby the contrast is increased or maximized.

In a further embodiment, the secondary radiation is detected exclusively in a subregion of the outer region of the detector. Thus, a local detection of the secondary radiation takes place. It is not determined globally across the entire detector. An optimization of the irradiation may thus take place based on just a local region in which the secondary radiation is detected.

According to a further embodiment, the automatic determination of the irradiation parameter takes place with the aid of an algorithm for machine learning. Thus, for example, a neural network may be trained to determine the irradiation parameter based on measurement values relating to the primary radiation and the secondary radiation. If the irradiation parameter is part of a multidimensional irradiation parameter vector, the use of the algorithm for machine learning is particularly advantageous because in this way, individual irradiation parameters (that is, vector components) may be calculated by simple means.

In a further embodiment, a dimension of an object causing the secondary radiation is determined dependent upon the detected primary radiation and the detected secondary radiation. For example, a thickness of the object may thus be determined very reliably. In this way, a double use of the detected primary radiation and the detected secondary radiation are the result, since therefrom not only the irradiation parameter, but also the dimension of the object may be determined.

In a further embodiment, a non-transitory computer readable medium having a computer program stored thereon is also provided, wherein the computer program includes instructions that, when the computer program is executed by a computer, cause the computer to carry out the method described above. The computer may be a control station of an imaging modality. The computer may have one or more processors and one or more storage elements. The program may be stored in the storage unit(s).

The above object is further achieved with an X-ray based imaging modality, in particular, a computed tomography system or a flat detector X-ray device with a processing circuit that is configured so that it carries out the method as set out above. The flat detector X-ray device may be realized as a fluoroscope, a radioscope and, in particular, as a C-arm device. The advantages and development possibilities set out above in relation to the method apply similarly also to the X-ray based imaging modality. Accordingly, the method features set out may be interpreted as functional features of the imaging modality.

In one embodiment of the X-ray based imaging modality, the X-ray based imaging modality has an output unit in order to output the irradiation parameter. For example, the irradiation parameter may be output visually on a display. If, for example, the irradiation parameter is the X-ray dose, then a user may monitor this X-ray dose. If the irradiation parameter includes, for example, the advice on whether an anti-scatter grid is useful or not, the user may follow the recommendation and utilize the anti-scatter grid in the beam path accordingly. The output unit may be configured such that the output unit outputs the irradiation parameter electronically so that it may be used for a subsequent arrangement.

### BRIEF DESCRIPTION OF THE DRAWINGS

The present disclosure is described in greater detail making reference to the accompanying drawings, in which:

FIG. 1 depicts an example of a C-arm angiography system with an industrial robot as a support apparatus.

FIG. 2 depicts an example of an X-ray recording with a directly irradiated and an indirectly irradiated region.

FIG. 3 depicts an example of the signal strength of the X-ray recording of FIG. 3 along a line.

FIG. 4 depicts an example of a schematic flow diagram.

### DETAILED DESCRIPTION

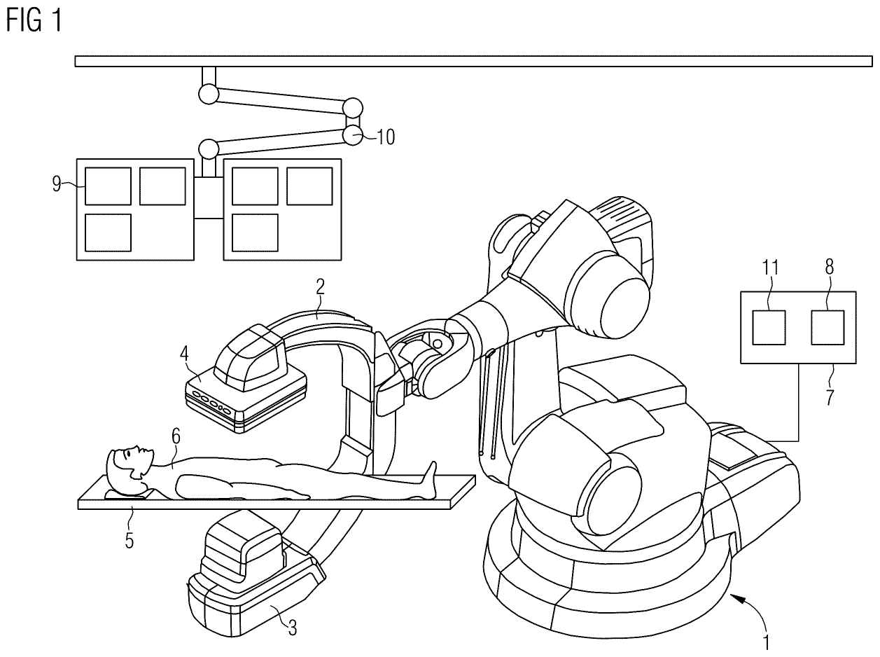

FIG. 1 shows, by way of example, a mono-planar X-ray system with a C-arm 2 held by a stand 1 in the form of a six-axis industrial or articulated arm robot at the respective ends of which an X-ray radiation source, for example, an X-ray emitter 3 with an X-ray tube and a collimator, and an X-ray image detector 4 as the image recording unit are mounted. The realization of the X-ray diagnostic facility is not dependent upon the industrial robot. Conventional C-arm devices may also be used.

Situated in the beam path of the X-ray emitter 3, on a tabletop 5 of a patient positioning table, is a patient 6 to be examined or a technical object as the examination object. Attached to the X-ray diagnostic facility is a system control unit 7 with a computing facility 8 for image processing, which receives and processes the image signals of the X-ray image detector 4 (for example, operating elements are not shown). The X-ray images may then be observed on displays of a monitor traffic lights set 9. The monitor traffic lights set 9 may be held by a ceiling-mounted, longitudinally movable, pivotable, rotatable, and height-adjustable carrier system 10 with a jib arm and a carrier arm that is capable of being lowered. Furthermore, an output unit 11 for output of an irradiation parameter may be provided in the system control unit 7.

In certain examples, a method for carrying out an X-ray imaging may be provided in which, in one embodiment, the X-ray dose may be controlled and/or regulated. In the following, in place of the two expressions “dose regulation” and “dose control,” for simplification, just the expression “dose regulation” is used. Whereas the identifying feature of regulation is a closed sequence of action (feedback) in which the control variable continuously influences itself in the action route of the control circuit, controlling is merely a process in which an input variable influences an output variable according to a particular law (see DIN IEC 60050-351).

A dose regulation may take place based on an integral detector signal P′ that is composed of a joint signal from a primary signal P and a secondary signal S. In a detector region of an X-ray detector, a pure primary radiation P is overlaid with the secondary radiation S, so that only the overlaid signal P′ may be measured as a detector signal. Each pixel element of a corresponding detector measures a detector signal P′ of this type.

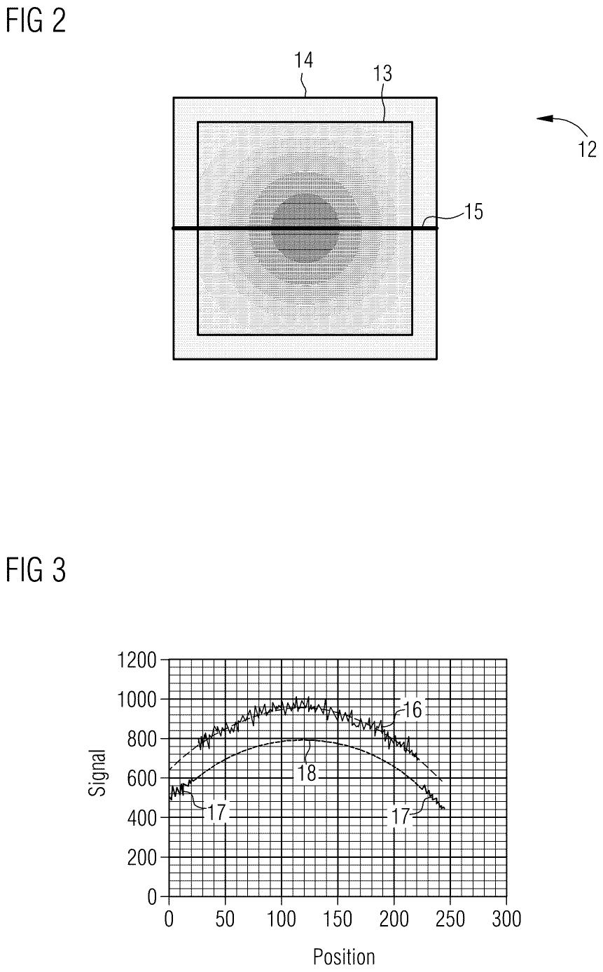

According to the example shown in FIG. 2, the X-ray detector 4 (detector, for short) has a detector surface 12 that is larger than a directly irradiated region 13. The directly irradiated region 13 is the region that the X-ray beam emerging from an X-ray radiation source meets without scattering. An outer region 14 on the detector surface 13 herein completely surrounds the directly irradiated region 13. Only secondary radiation, which arises by way of scattering of X-ray radiation at an object to be examined (e.g., a patient 6) and is undirected, is incident in the outer region 14.

The outer region 14 may be created, for example, in that an aperture (collimator) that at least substantially blocks the X-ray radiation from the outer region 14 is placed in the beam path. In the present example of FIG. 2, the aperture may be a rectangular and/or square opening. With this aperture, the entire detector surface 12 may not be irradiated with the primary radiation (i.e., X-ray radiation from the X-ray source attenuated by the object). An outer region 14 not irradiated by the primary radiation may also result therefrom that the detector (in particular, a flat detector) is already structurally larger than the maximum clinically used region of the system.

The signal in the outer region 14 (that is, the pixel signals there) outside the directly irradiated region 13 is therefore primarily attributable to scattered radiation and/or secondary radiation S. A possible primary transparency of the aperture (that is, of the collimator) may be eliminated from the signal experimentally or theoretically.

The outer region 14 may be situated on only one side of the directly irradiated region 13. However, it may also be situated on a plurality of sides, for example, on two mutually opposite sides of the directly irradiated region 13. Furthermore, it may also be situated on three sides or four sides, wherein in FIG. 2, the last example is shown.

The pixels in the outer region 14 may be used to interpolate the signal course of the scattered radiation over the entire detector surface. FIG. 3 shows an example of this. It is the pixels that lie on the line 15 in FIG. 2 that are evaluated here. In the directly irradiated region 13, a signal strength over the position in accordance with the curve 16 results. In the not directly irradiated region and/or the outer region 14, the curve 17 for the signal strength is formed.

In the present example, the curve 17 has two curve portions: one in the left image half and one in the right image half. Both curve portions may be used, by way of interpolation, to determine the scattered radiation along the entire line 15. For example, the interpolation may be carried out with a polynomial.

In the example of FIG. 2, a scattering body was used as a phantom. This results, in FIG. 3, in the arc-shaped course 18 that shows a scattered radiation maximum approximately in the image center. Since the scattered radiation has an arc-shaped intensity course of this type, the signal course of the primary radiation P overlaid with the secondary radiation S is also, on average, arc-shaped according to the curve 16. If no scattered radiation is present, the course of the curve 16 may be flat and/or linear, since the primary radiation signal may accordingly be calibrated so that contrast values (for example, of bone) are substantially the same at the image edge and in the image center. From FIG. 3, it may therefore be seen that, in the directly irradiated region 13, the primary radiation S is detected overlaid with the secondary radiation S.

The scattered radiation signal, (i.e., the signal resulting from the secondary radiation S in the outer region 14), may be placed in relation to the signal from the directly irradiated region 13. Thus, the ratio of the primary and the scattered radiation may be optimized. For example, the ratio may be maximized within the technical limits. By this, for example, the image contrast may also be maximized.

With the detected primary radiation (possibly overlaid with the secondary radiation) and the secondary radiation detected in the outer region, a dose regulation for the X-ray imaging may be realized. Thus, for example, the scattered radiation may be used to correct the signals in the directly irradiated region, so that a dose regulation may be optimized solely in relation to the signal for the primary radiation. In this way, for example, a true water value may be determined.

The signal from the scattered radiation (for short: scatter signal; made up from many pixel signals together) may be evaluated locally for the dose regulation. This means that the outer region 14, which is not directly irradiated, does not have to be evaluated in its entirety. For example, only the left upper corner of the outer region 14 of FIG. 2 is evaluated with regard to the scatter signal, so that the dose regulation may be optimized in relation thereto. In the example of FIG. 2, the outer region 14 forms an “anti-scatter frame” around the primary image and/or the directly irradiated region 13. This anti-scatter frame contains “long range” positional information regarding the scatter intensity, since the projection location of a scatter center may be far removed from the actual detection location of the scattered radiation.

As shown in relation to FIG. 3, the “long range” scatter signal may be calculated into the image field homogenization. A global or position-dependent correction of the scattered and/or secondary radiation may be undertaken. The image contrast may thereby be increased.

By way of knowledge of the primary radiation P and the secondary radiation S, and also knowledge of the technical properties of an available anti-scatter grid and/or filter, it may be calculated whether the use of the grid/filter brings about an image improvement, possibly dependent upon the radiation dose.

From the detected signal values relating to the primary radiation and the secondary radiation, an irradiation parameter may be automatically determined, which may be interpreted as a recommendation of whether an anti-scatter grid is useful or not. This decision regarding whether the grid delivers an image quality improvement or not depends upon the thickness of the object and/or the patient. Specifically for pediatric or extremity applications, it may be useful to remove or dispense with the grids.

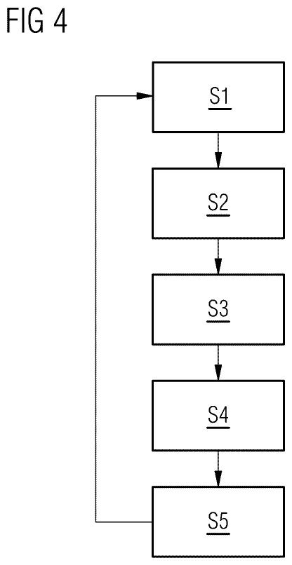

FIG. 4 shows an embodiment of a method sequence in a block diagram. In act S1, a detection of a primary radiation takes place overlaid with a secondary radiation in a region of the detector directly irradiated by the X-ray radiation. The detection means that corresponding signal values that the primary radiation has caused are measured or are obtained from a memory store.

In act S2, a detection of a secondary radiation in an outer region of the detector outside of the directly irradiated region takes place. Here also, corresponding signal values are measured and/or obtained or recorded with regard to the secondary radiation.

In act S3, an automatic determination of an irradiation parameter takes place dependent upon the detected primary radiation and the detected secondary radiation. The irradiation parameter may relate to any desired body-related and/or physical value of an X-ray imaging modality that may influence the irradiation or the resultant image.

In optional act S4, an optimization of the irradiation parameter may be undertaken. If the irradiation parameter is, for example, an X-ray dose, this may be optimized and/or minimized as far as a useful limit.

In act S5, the carrying out of the X-ray imaging then takes place based on the irradiation parameter. Herein, an X-ray recording is obtained based on the automatically determined and/or optimized irradiation parameter.

In an advantageous manner, it is possible to adapt and optimize a dose regulation with regard to resultant image quality and/or with regard to resulting image contrast.

A further advantage of the separate detection of primary radiation and secondary radiation in the form of two values includes that apart from the automatic determination of an irradiation parameter, a reliable estimation of the true object and/or patient thickness is also enabled.

It is furthermore advantageous that the method described may also be applied to non-collimated images if, despite the image field being expanded to the maximum clinically possible, a (small) edge region of the detector still remains that sees only the scatter signal.

Overall, it is an advantage of the disclosure that a particular X-ray dose may be better utilized. Furthermore, the image processing may be optimized since more reliable primary absorption values (water values) may be made use of. Furthermore, the contrast and/or noise suppression may be optimized to particular signal values (gray-scale regions).

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for carrying out an X-ray imaging on a basis of X-ray radiation, the method comprising:
detecting a primary radiation overlaid with secondary radiation in a region of a detector directly irradiated by the X-ray radiation;
detecting a secondary radiation in an outer region of the detector outside of the directly irradiated region;
automatically determining an irradiation parameter dependent upon the detected primary radiation and the detected secondary radiation; and
carrying out the X-ray imaging based on the irradiation parameter.

2. The method of claim 1, wherein the irradiation parameter relates to an X-ray dose.

3. The method of claim 2, wherein the X-ray dose for the X-ray imaging is regulated by way of an automatic dose regulation.

4. The method of claim 1, wherein the irradiation parameter relates to a presence of an anti-scatter grid in a beam path of the X-ray radiation, and
wherein the automatically determining of the irradiation parameter takes into account an effect of the anti-scatter grid on the X-ray radiation.

5. The method of claim 1, wherein the irradiation parameter is iteratively determined such that an image quality is optimized in the X-ray imaging.

6. The method of claim 1, wherein a ratio between the detected primary radiation and the detected secondary radiation in relation to an optimization criterion is optimized or maximized in the automatically determining of the irradiation parameter.

7. The method of claim 1, wherein the automatically determining of the irradiation parameter comprises:
determining a pure component of the primary radiation, wherein the secondary radiation is interpolated for the directly irradiated region from the detected secondary radiation in the outer region outside the directly irradiated region; and
subtracting the interpolated secondary radiation from the detected overlaid primary radiation.

8. The method of claim 7, wherein the irradiation parameter relates to an X-ray dose,
wherein the X-ray dose for the X-ray imaging is regulated by way of an automatic dose regulation, and
wherein the pure component of the primary radiation represents a control variable in the automatic dose regulation.

9. The method of claim 7, wherein, in the carrying out of the X-ray imaging, an X-ray image is obtained only based on the pure component of the primary radiation.

10. The method of claim 1, wherein the secondary radiation is detected exclusively in a subregion of the outer region of the detector.

11. The method of claim 1, wherein the automatically determining of the irradiation parameter takes place with aid of an algorithm for machine learning.

12. The method of claim 1, further comprising:
determining a dimension of an object causing the secondary radiation dependent upon the detected primary radiation and the detected secondary radiation.

13. A non-transitory computer readable medium having a computer program that contains instructions that, when executed by a computer, cause the computer to:
detect a primary radiation overlaid with secondary radiation in a region of a detector directly irradiated by X-ray radiation;
detect a secondary radiation in an outer region of the detector outside of the directly irradiated region;
automatically determine an irradiation parameter dependent upon the detected primary radiation and the detected secondary radiation; and
carry out an X-ray imaging based on the irradiation parameter.

14. An X-ray based imaging modality comprising:
at least one processor configured to:
detect a primary radiation overlaid with secondary radiation in a region of a detector directly irradiated by X-ray radiation;
detect a secondary radiation in an outer region of the detector outside of the directly irradiated region;
automatically determine an irradiation parameter dependent upon the detected primary radiation and the detected secondary radiation; and
carry out the X-ray imaging based on the irradiation parameter.

15. The X-ray based imaging modality of claim 14, further comprising:
an output unit configured to output the irradiation parameter.

16. The X-ray based imaging modality of claim 14, wherein the X-ray based imaging modality is a computed tomography system or a flat detector X-ray device.
